(19) United States
(12) Patent Application Publication
(10) Pub. No.: US 2025/0295602 A1
(43) Pub. Date: Sep. 25, 2025

(54) FLOW PATH STRUCTURE, FLOW PATH STRUCTURE UNIT, AND METHOD FOR PRODUCING LIPID PARTICLES

(71) Applicant: Kabushiki Kaisha Toshiba, Tokyo (JP)

(72) Inventors: Masato AKITA, Kawasaki (JP); Mitsuaki KATO, Kawasaki (JP)

(73) Assignee: Kabushiki Kaisha Toshiba, Tokyo (JP)

(21) Appl. No.: 19/045,660

(22) Filed: Feb. 5, 2025

(30) Foreign Application Priority Data
Mar. 19, 2024 (JP) 2024-044245

(51) Int. Cl. A61K 9/50

(52) U.S. Cl. CPC A61K 9/5089; A61K 9/5015

(57) ABSTRACT

A flow path structure according to an embodiment includes: a first flow path, a second flow path, and a third flow path. The second flow path is connected to the first flow path, and the third flow path is connected to the first flow path and the second flow path. When a distance between a top surface and a bottom surface of a flow path is defined as a flow path depth, the flow path depth of the second flow path at an opening where the first flow path is connected to the second flow path is not constant, and the maximum value of the flow path depth of the second flow path at the opening is smaller than a flow path depth of the first flow path.

### CROSS-REFERENCE TO RELATED APPLICATION

This application is based upon and claims the benefit of priority from Japanese Patent Application No. 2024-044245, filed Mar. 19, 2024, the entire contents of which are incorporated herein by reference.

### FIELD

Embodiments of the present invention relate generally to a flow path structure, a flow path structure unit, and a method for producing lipid particles.

### BACKGROUND

In order to uniformly progress a chemical reaction in a liquid, it is necessary to forcibly promote mixing of reactants by an eddy or a turbulent flow caused by stirring. Therefore, a mixing method for further promoting mixing of two liquids is required.

### DESCRIPTION OF THE DRAWINGS

FIG. 1 is a perspective view illustrating an example of a flow path structure according to a first embodiment;

FIG. 2 is a schematic view illustrating an example of the flow path structure in the first embodiment;

FIG. 3 is a plan view illustrating an example of the flow path structure in the first embodiment;

FIG. 4 is a diagram illustrating an example of a simulation result of a state of mixing of two liquids;

FIG. 5 is a schematic view illustrating a state of mixing of two liquids;

FIG. 6 is an example of a cross-sectional view along a main flow direction of a first flow path and a third flow path of the flow path structure according to the first embodiment;

FIG. 7 is an example of a cross-sectional view along a main flow direction of a first flow path and a third flow path of a flow path structure in a first modification;

FIG. 8 is a schematic view illustrating a state of mixing of two liquids in the first modification;

FIG. 9 is an example of a cross-sectional view along the main flow direction of a first flow path and a third flow path of a flow path structure in a second modification;

FIG. 10 is a schematic view illustrating a variation of a flow path structure 100 in a third modification;

FIG. 11 is an example of a plan view of a flow path structure according to a second embodiment;

FIG. 12 is an example of a schematic view of a flow path structure unit;

FIG. 13 is an example of a schematic view of the flow path structure unit;

FIG. 14 is an example of a schematic view of the flow path structure unit;

FIG. 15 is an example of a schematic view of a method for producing the flow path structure;

FIG. 16 is a diagram illustrating an example of a lipid particle of a sixth embodiment;

FIG. 17 is a flowchart illustrating an example of a method for producing lipid particles; and

FIG. 18 is a view illustrating an example of a flow path structure used in the method for producing lipid particles of the sixth embodiment.

### DETAILED DESCRIPTION

A flow path structure according to an embodiment includes: a first flow path, a second flow path, and a third flow path. The second flow path is connected to the first flow path, and the third flow path is connected to the first flow path and the second flow path. When a distance between a top surface and a bottom surface of a flow path is defined as a flow path depth, the flow path depth of the second flow path at an opening where the first flow path is connected to the second flow path is not constant, and the maximum value of the flow path depth of the second flow path at the opening is smaller than a flow path depth of the first flow path.

Hereinafter, embodiments will be described with reference to the accompanying drawings. In each embodiment, substantially the same components will be given the same reference numerals, and the description thereof will be partially omitted. The drawings are schematic, and the relationship between the thickness and the planar dimension of each portion, the ratio of the thickness of each portion, and the like may be different from actual ones.

When the fluid flows through the flow path structure, the description will be given on the assumption that the direction of the main flow of the flow in the flow path is as indicated by an arrow in the drawing and is substantially along the tube axis direction.

In addition, in the present specification, the “flow path” refers to a space that is formed inside the flow path structure and through which a fluid can flow. The flow path has openings on the upstream side and the downstream side of the fluid, respectively. The flow path has a base material such as resin, glass, ceramics, or metal as a wall surface, and a top surface or a bottom surface of the flow path is sealed by the base material of the flow path structure. In the present specification, a liquid will be described as an example of the fluid.

### First Embodiment

FIG. 1 is a perspective view illustrating an example of a flow path structure 100 according to a first embodiment. In the present embodiment, the flow path structure includes a first flow path 1, a second flow path 2 connected to the first flow path 1, and a third flow path 3 connected to the first flow path 1 and the second flow path 2. In the present specification, the second flow path 2 is indicated by dot hatching. When the distance between the top surface and the bottom surface of a flow path is defined as the flow path depth, the flow path depth of the second flow path 2 at the opening where the first flow path 1 is connected to the second flow path 2 is not constant. The maximum value (h2) of the flow path depth of the second flow path 2 in the opening is smaller than the flow path depth (h1) of the first flow path 1. An arrow in the drawing indicates a direction of a main flow of the liquid flowing inside the flow path structure. The liquids flowing in the first flow path 1 or the second flow path 2 merge and flow in the third flow path 3. In the present embodiment, when the liquid flows through the flow path structure, the downstream side of the second flow path 2 is connected to the downstream side of the first flow path 1, and the third flow path 3 is connected to the further downstream side of the first flow path 1 and the second flow path 2. Here, “the flow path is connected” refers to a state where an end portion of the flow path is liquid-tightly connected to another flow path to form a continuous space such that an internal space of the flow path communicates with an internal space of another flow path. A material of flow path structure 100 is not particularly limited, and is, for example, a solid resin such as cycloolefin polymer (COP). The material of the flow path structure 100 will be described later in detail. The first flow path 1, the second flow path 2, and the third flow path 3 are spaces formed by cutting a bulk solid such as COP to form grooves.

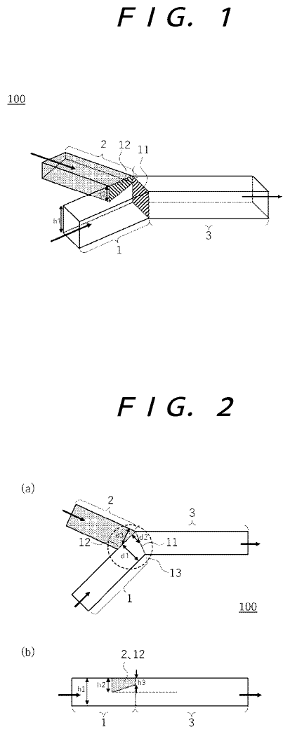

FIG. 2(a) is a plan view illustrating an example of the flow path structure 100 in the first embodiment. In the present specification, unless otherwise specified, in FIG. 2(a), a flow path wall surface positioned closest to the paper surface is described as a top surface, a flow path wall surface facing the top surface and positioned on a deeper side of the paper surface than the top surface is described as a bottom surface, and a flow path wall surface intersecting with the top surface and the bottom surface is described as a flow path side surface. In addition, the dimension of the flow path perpendicular to the paper surface, that is, the distance between the top surface and the bottom surface will be described as the flow path depth. The flow path depth is based on a plane including the top surface unless otherwise specified. In the present specification, unless otherwise specified, a dimension in a direction perpendicular to the tube axis direction and parallel to the paper surface in FIG. 2(a) will be described as a flow path width. As illustrated in FIGS. 1 and 2, the top surfaces of the first flow path 1, the second flow path 2, and the third flow path 3 are preferably included in a single plane. This is because the flow path structure 100 can be easily and accurately manufactured. Details are described in the following embodiments.

The flow path widths and the flow path depths of the first flow path 1, the second flow path 2, and the third flow path 3 are appropriately determined in consideration of various conditions such as the type and flow velocity of the liquid supplied to the flow path structure. For example, there is a Reynolds number Re as a numerical value in consideration of the shape of the flow path structure 100, the type and flow velocity of the liquid, and the like. The Reynolds number Re is a dimensionless numerical value defined by the following Formula (1) using ρ [kg/m3] indicating the density of liquid, V [m/s] indicating the velocity of liquid, L [m] indicating the representative length, and μ [Pa·s] indicating the viscosity of liquid.

Re=ρVL/ (1)

In the calculation of the Reynolds number of the flow path according to the present embodiment, the hydraulic diameter dH is set as the representative length L. The hydraulic diameter dH is defined by the following Formula (2), where A is a flow path cross-sectional area and P is a flow path cross-sectional edge length.

dH=4A/P (2)

The representative length L for obtaining the Reynolds number of the liquid flowing in the flow path is often the hydraulic diameter dH, but the flow path depth, the flow path width, or an average value thereof may be used as the representative length.

In order to exert the effect of the present invention, the Reynolds number calculated from the above Formula (1) is preferably 10 or more in the flow path structure. In addition, the Reynolds number is preferably less than 2,300 in the flow path structure in order to generate uniform vortices and avoid the generation of turbulence in the flow path. The Reynolds number is preferably less than 2,300 also at a location where the second flow path 2 merges with the first flow path 1 where the area of the flow path cross section perpendicular to the tube axis direction is the smallest in the drawing. In order to further promote the generation of uniform vortices and further prevent the generation of turbulent flows in the flow path, the Reynolds number in the flow path structure is more preferably approximately 50 or more and 1,000 or less. The cross-sectional area of the flow path, included in flow path structure 100, perpendicular to the tube axis direction is preferably 100 mm² or less. However, from the viewpoint of suppressing cavitation in the flow path and avoiding clogging due to bubbles or the like in the flow path, the flow path width and the flow path depth are preferably 5 μm or more, and since the accuracy of die molding or cutting processing, which is a method for manufacturing the flow path structure 100, is generally 5 μm, in consideration of the accuracy in flow path machining, the flow path width and the flow path depth are preferably 10 μm or more. The flow path depth is preferably equal to or less than the flow path widths of the first flow path 1, the second flow path 2, and the third flow path 3 from the viewpoint of securing the strength of a mold for manufacturing the flow path structure 100.

Hereinafter, each portion of the flow path structure in the present embodiment will be described in detail.

In the first flow path 1, the flow path width and the flow path depth are constant in most regions. However, the present invention is not limited thereto in a mixing region 13 near the merging portion of first to third flow paths. For example, at the location where the second flow path 2 is connected to the first flow path 1, when the surface surrounded by the ridge line of the end portion of the second flow path 2 is defined as a second opening 12, the shortest distance (d1 in the drawing) between the second opening 12 and the flow path side surface of the first flow path 1 facing the second opening 12 is defined as the flow path width of the first flow path 1. In the boundary between the first flow path 1 and the third flow path 3, when the surface surrounded by the ridge line of the end portion of the first flow path 1 is defined as a first opening 11, the shortest distance (d2 in the drawing) from any point of the first opening 11 to the second opening 12 is defined as the flow path width of the first flow path 1. The flow path width defined by the first opening 11 and the second opening 12 decreases toward the downstream side. The flow path width and d1 illustrated in the drawing in the first flow path 1 other than the mixing region 13 illustrated in the present embodiment are, for example, 0.3 mm, and the flow path depth is, for example, 0.3 mm. The average flow velocity of the liquid flowing through the first flow path 1 is preferably approximately 0.1 m/s or more. Assuming that the liquid is close to water and the representative length is the hydraulic diameter, the Reynolds number at this time is around 30 around room temperature. When a pump is used as a device for allowing a liquid to flow into the flow path structure according to the present embodiment, it is preferable to use a pump that does not cause pulsation. As such a pump, a pump having a liquid feeding amount of approximately 1 mL/sec can be easily obtained. In consideration of this circumstance, the first flow path 1 is preferably a so-called micro flow path having a flow path width and a flow path depth of approximately 3 mm or less. The first flow path 1 may be described as a main flow path in the flow path structure 100.

The second flow path 2 has a constant flow path width in most regions. However, this is not the case in the mixing region 13. For example, at the location where the second flow path 2 is connected to the first flow path 1, the shortest distance (d3 in the drawing) between the second opening 12 and the flow path wall surface of the second flow path 2 connected to the third flow path 3 is defined as the flow path width of the second flow path 2. The flow path width of the second flow path 2 other than the mixing region 13 illustrated in the present embodiment is, for example, 0.3 mm.

At the location where the second flow path 2 is connected to the first flow path 1, the shortest distance from any point of the second opening 12 to the flow path wall surface of the second flow path 2 farther from the first flow path 1 is defined as the flow path width of the second flow path 2. The flow path width defined by the second opening 12 and the side surface of the second flow path 2 decreases toward the downstream side.

Furthermore, the second flow path 2 has a different flow path depth depending on the position. FIG. 2(b) is an example of a cross-sectional view along a main flow direction of a first flow path 1 and a third flow path 3 of the flow path structure 100 according to the first embodiment. As described above, the second flow path 2 is indicated by dot hatching. The maximum flow path depth (h2) of the second flow path 2 is equal to or less than the flow path depth (h1) of the first flow path 1. The depth of the second flow path 2 at the location where the second flow path 2 is connected to the first flow path 1 changes along the width direction of the second flow path 2. FIG. 2(b) exemplifies a case where the flow path depth of the second flow path 2 changes at a constant inclination, and the flow path depth (h2) of the second flow path 2 at the location where the flow path side surface of the second flow path 2 and the flow path side surface of the first flow path 1 are in contact with each other is larger than the flow path depth (h3) of the second flow path 2 at the flow path wall surface on the opposite side with the second flow path 2 interposed therebetween at the location where the flow path side surface of the second flow path 2 and the flow path side surface of the first flow path 1 are in contact with each other. That is, the depth of the second flow path 2 at the location where the second flow path 2 is connected to the first flow path 1 becomes shallower toward the flow path wall surface on the opposite side. As a result, since the flow path cross-sectional area is reduced, it is possible to increase the flow velocity of the passing liquid. The maximum value (h2) of the flow path depth of the second flow path 2 is preferably ½ or less of the flow path depth (h1) of the first flow path, and more preferably ⅓ or less. That is, in flow path structure 100 illustrated in the present embodiment, in a case where the flow path depth of first flow path 1 is, for example, 0.3 mm, the maximum value (h2) of the depth of second flow path 2 is preferably 0.15 mm or less, and more preferably 0.10 mm or less. However, as described in the description of the first to third flow paths, the maximum value (h2) of the depth of the second flow path 2 is preferably 5 μm or more, and more preferably 10 μm or more from the viewpoint of manufacturing of the flow path and practical use. The minimum value (h3) of the depth of the second flow path 2 may be smaller than the maximum value, and may be 0 mm (a state where the cross-sectional shape of the second flow path 2 in FIG. 2(b) is a triangle). The flow path width of the second flow path 2 illustrated in the present embodiment is, for example, 0.3 mm, the maximum value of the flow path depth is 0.1 mm, and the minimum value of the flow path depth is 0.0 mm. The average flow velocity of the liquid flowing through the second flow path 2 is preferably approximately 0.1 m/s or more. Assuming that the liquid is close to water and the representative length is the hydraulic diameter, the Reynolds number at this time is around 30 around room temperature. When a pump is used as a device for allowing a liquid to flow into the flow path structure according to the present embodiment, it is preferable to use a pump that does not cause pulsation. As such a pump, a pump having a liquid feeding amount of approximately 1 mL/sec can be easily obtained. In consideration of this circumstance, the second flow path 2 is preferably a so-called micro flow path having a flow path width and a flow path depth of approximately 3 mm or less. The second flow path 2 may be described as a sub flow path in the flow path structure 100.

In the third flow path 3, the flow path width and the flow path depth may be constant in most regions. However, this is not the case in the mixing region 13. For example, at the location where the first flow path 1 is connected to the third flow path 3, the shortest distance from any point of the first opening 11 to the flow path wall surface of the third flow path 3 connected to the second flow path 2 is defined as the flow path width of the third flow path 3. The flow path width defined by the first opening 11 and the side surface of the third flow path 3 increases toward the downstream side. The flow path of the third flow path 3 illustrated in the present embodiment is, for example, 0.3 mm, and the flow path depth is 0.3 mm. The average flow velocity of the liquid flowing through the third flow path 3 is preferably approximately 0.1 m/s or more. Assuming that the liquid is close to water and the representative length is the hydraulic diameter, the Reynolds number at this time is around 30 around room temperature. When a pump is used as a device for allowing a liquid to flow into the flow path structure according to the present embodiment, it is preferable to use a pump that does not cause pulsation. As such a pump, a pump having a liquid feeding amount of approximately 1 mL/sec can be easily obtained. In consideration of this circumstance, the third flow path 3 is preferably a so-called micro flow path having a flow path width and a flow path depth of approximately 3 mm or less. The third flow path 3 may be described as a merged flow path in the flow path structure 100.

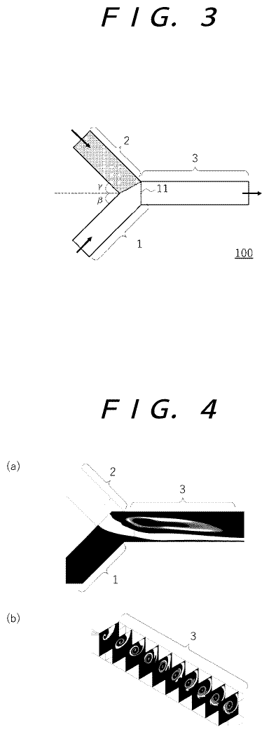

FIG. 3 is a plan view illustrating an example of the flow path structure 100 according to the first embodiment, and an angle formed by the first flow path and the second flow path will be described with reference to this view. As also illustrated in FIG. 3, in the flow path structure 100 in the first embodiment, the tube axis direction of the first flow path 1 and the tube axis direction of the third flow path 3 do not align with each other. That is, an example in which the first flow path 1 and the third flow path 3 are not continuous straight lines is illustrated. When the first flow path 1 is connected such that the flow path directions of the third flow path 3 intersect with each other, in the third flow path 3, a liquid (a flow from the main flow path) passing through the first flow path 1 and a liquid (a flow from the sub flow path) passing through the second flow path 2 form a vortex and can be quickly mixed. Note that the first flow path 1 and the second flow path 2 are symmetrical with respect to the third flow path 3, and when an angle formed by a side surface of the first flow path 1 and a side surface of the third flow path 3 is β and an angle formed by a side surface of the second flow path 2 and a side surface of the third flow path 3 is γ, β and γ may be different from each other, but β and γ are preferably equal to each other. By setting the values of β and γ to a predetermined value exceeding 0° or more, it is preferable to prevent the flow from the main flow path and the flow from the sub flow path from merging in substantially parallel and maintain the mixing speed in the third flow path 3. Specifically, when the second flow path 2 perpendicularly merges with the first flow path 1 (that is, the sum of β and γ is) 90°, the opening area of the second opening 12 is reduced, and thus the mixing speed is the fastest. In addition, by setting the values of β and γ to predetermined values or less, it is preferable to prevent the flow from the main flow path and the flow from the sub flow path from facing each other and colliding with each other, to assist generation of orderly vortices, and to maintain uniformity of mixing. Therefore, it is preferable that the first flow path 1 and the second flow path 2 intersect with each other at an angle within a predetermined range. Therefore, the sum of β and γ is preferably 60° or more and 120° or less, and the sum of β and γ is particularly preferably 90°. Furthermore, it is more preferable that the first flow path 1 and the second flow path 2 are symmetrical with respect to the third flow path 3. Therefore, each of β and γ is preferably 30° or more and 60° or less, and particularly preferably β=γ=45°. Note that, when the liquid flows in the flow path structure 100 described here, an angle formed by the main flow direction of the liquid flowing in the first flow path 1 and the main flow direction of the liquid flowing in the third flow path 3 corresponds to β, and an angle formed by the main flow direction of the liquid flowing in the second flow path 2 and the main flow direction of the liquid flowing in the third flow path 3 corresponds to.

As described above, since the flow path structure 100 has a structure in which the flow path depth of the sub flow path changes along the flow direction in the main flow path, the liquid passing through the main flow path and flowing through the merged flow path and the liquid passing through the sub flow path and flowing through the merged flow path are quickly and efficiently mixed, and the mixing of the two liquids can be promoted. This can also be achieved with a relatively simple structure.

FIG. 4 is a diagram illustrating an example of a simulation result of a state of mixing of two liquids. FIG. 4(a) is a diagram when the flow path structure 100 is viewed from the top surface side, and FIG. 4(b) is a diagram illustrating the mixing degree at each point when the liquid flowing through the third flow path 3 is sliced perpendicularly to the main flow direction. The flow from the first flow path 1 is illustrated in black, and the flow from the second flow path 2 is illustrated in white. As can be seen from FIGS. 4(a) and 4(b), a vortex is generated in the third flow path 3, and the two liquids are mixed.

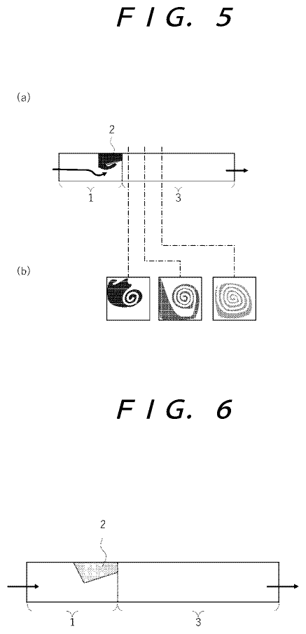

FIG. 5 is a schematic view illustrating a state of mixing of two liquids. FIG. 5(a) is an example of a cross-sectional view along the main flow direction of the first flow path 1 and the third flow path 3 of the flow path structure 100 in the first embodiment. The liquid (hereinafter referred to as “liquid A”) passing through the second flow path 2 is indicated by hatching, and the main flow direction of the liquid passing through the first flow path 1 and the third flow path 3 is indicated by an arrow. Since the liquid A has a wide step when connected from the second flow path 2 to the first flow path 1, flow separation occurs below the step. Since this separation moves while being stretched downstream by the flow of the first flow path 1, a structure of a coaxial small vortex is generated downstream where the two flows merge. Furthermore, a large vortex (what is called a general swirl) generated by the two mergers reinforces the structure of the small vortex more stably. Since the first small vortex tends to be generated in the direction of the step, it is advantageous to generate the vortex on the downstream side in order to stretch the structure of the vortex downstream. Therefore, in the second flow path 2, the main flow path side of the upstream side sub flow path of the first flow path 1 needs to be deep and the flow width needs to be large. In such a structure, as compared with a case where the bottom surface of the second flow path 2 is uniformly shallow in the flow path depth of the flow path structure 100 and the top surface and the bottom surface are parallel, stretching of small vortices is smooth, and vortices due to the liquid A immediately after merging and the liquid (hereinafter referred to as “liquid B”) passing through the first flow path 1 are ordered. In addition, since there is a limit to vortices that can be generated with a limited flow path width, when the flow rate of the liquid A becomes extremely high, the liquid A is not caught in the vortex, which hinders rapid mixing. However, by reducing the depth of the second flow path 2 on the downstream side of the first flow path 1, the flow rate of the liquid A flowing from the second flow path 2 into the first flow path 1 can be suppressed to the extent that the liquid A can be caught in the vortex. FIG. 5(b) is a cross-sectional view of the flow path structure 100 perpendicular to the main flow direction of the third flow path 3. The position of the broken line in FIG. 5(a) and the position of the cross-sectional view in FIG. 5(b) are described for easy understanding of the concept of mixing, and the actual degree of mixing may vary depending on various conditions such as liquid properties, temperature, flow velocity, and dimensions of the flow path. The liquid A immediately after merging with the first flow path 1 is swept away while being wound with a vortex, whereby a substantially multi-layer concentric vortex as illustrated in the drawing is formed in the third flow path 3. In the flow path structure 100 illustrated in the present embodiment, since the ratio of the substantially multi-layer concentric vortex in the third flow path 3 as illustrated in the drawing is large and the amount of liquid not constituting the vortex is relatively small, the liquid A and the liquid B are quickly mixed.

Note that the liquid A and the liquid B are not necessarily the same type of liquid, and may have different properties including viscosity, temperature, flow velocity, and the like. The shape of the bottom surface of the second flow path 2 may be designed linearly or curvilinearly in accordance with the properties of these two liquids.

As described above, the flow path structure 100 having the structure in which the main flow path, the sub flow path, and the merged flow path are included and the depth of the sub flow path changes along the width direction of the sub flow path can quickly and efficiently mix the flow from the main flow path and the flow from the sub flow path in the merged flow path, and the mixing of the two liquids can be further promoted. This can also be achieved with a relatively simple structure.

The flow path structure 100 may be a tubular structure made of resin such as acrylic, polyethylene glycol (PEG) ethylene, polypropylene, or polycarbonate, glass, ceramics, or metal, or may be a flow path structure embedded in a solid such as resin (for example, acrylic, polyethylene, polypropylene, or polycarbonate), glass, ceramics, or metal. In a case where the flow path structure 100 is embedded in a solid, the inflow port of the liquid upstream of the first flow path 1 and the second flow path 2 and the outflow port of the liquid downstream of the third flow path 3 communicate with the outside of the solid.

The top surfaces of the first flow path 1, the second flow path 2, and the third flow path 3 are preferably included in a single plane. When the top surface of flow path structure 100 is formed of one flat plate, the flow path can be easily sealed by forming the flow path by pressing or cutting using a mold and then covering the top surface from above.

The side surface of the second flow path 2 is not necessarily perpendicular to the top surface. FIG. 6 is an example of a cross-sectional view along the main flow direction of the first flow path 1 and the third flow path 3 of the flow path structure 100 in the first embodiment. As illustrated in FIG. 6, by making the side surface of the second flow path 2 positioned on the upstream side of the flow in the first flow path 1 oblique to the side surface of the second flow path 2 positioned on the downstream side of the flow in the first flow path 1, the flow path structure 100 can be easily manufactured, and mass productivity can be improved.

In the first embodiment, the example in which the flow path widths of the first flow path 1, the second flow path 2, and the third flow path 3 are constant has been described, but the embodiment of the present invention is not limited thereto, and the flow path width may change from upstream to downstream.

In the present embodiment, by further adjusting the flow velocities of the first flow path 1 and the second flow path 2, the ratio of the flow that does not form a vortex in the third flow path 3 may be further suppressed.

### First Modification

Components common to those of the first embodiment will be given the same reference numerals, and modifications will be described below while omitting description of components having the same configuration and function.

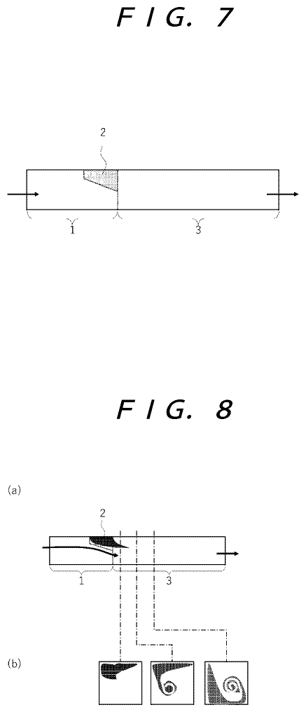

FIG. 7 is an example of a cross-sectional view along the main flow direction of the first flow path 1 and the third flow path 3 of the flow path structure 100 in the present modification. Unlike the first embodiment, the depth of the second flow path 2 at the location where the second flow path 2 is connected to the first flow path 1 becomes deeper toward the third flow path 3. The flow path width of the second flow path 2 illustrated in the present modification is 0.3 mm, the maximum value of the flow path depth is 0.1 mm, and the minimum value of the flow path depth is 0.0 mm. At this time, the average flow velocity of the liquid flowing through the second flow path 2 is preferably approximately 0.1 m/s or more. Assuming that the liquid is close to water and the representative length is the hydraulic diameter, the Reynolds number at this time is around 30 around room temperature. The second flow path 2 may be described as a sub flow path in the flow path structure 100.

FIG. 8 is a schematic view illustrating how two liquids are mixed in the present modification. FIG. 8(a) is an example of a cross-sectional view along the main flow direction of the first flow path 1 and the third flow path 3 of the flow path structure 100 in the present modification. The liquid A is indicated by hatching, and the main flow direction of the liquid passing through the first flow path 1 and the third flow path 3 is indicated by an arrow. In the second flow path 2, since the flow path depth on the upstream side of the liquid A is small, the small vortex caused by the liquid A is swept away while winding the vortex from the upstream to the downstream of the liquid B while being distorted by the flow of the liquid B. FIG. 8(b) is of flow path structure 100 a cross-sectional view perpendicular to the main flow direction of third flow path 3 in the present modification. Since a vortex is formed, mixing of the two liquids can be promoted.

### Second Modification

Components common to those of the first embodiment will be given the same reference numerals, and modifications will be described below while omitting description of components having the same configuration and function.

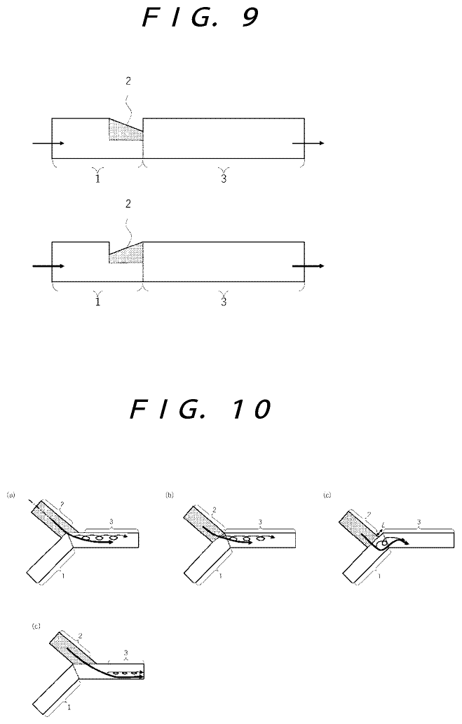

FIG. 9 is an example of a cross-sectional view along the main flow direction of the first flow path 1 and the third flow path 3 of the flow path structure 100 in the present modification. The second flow path 2 is indicated by hatching. As illustrated in the drawing, the distance between the top surface and the bottom surface of the second flow path 2, that is, the flow path depth may be changed by inclining the top surface of the second flow path 2.

### Third Modification

Components common to those of the first embodiment will be given the same reference numerals, and modifications will be described below while omitting description of components having the same configuration and function.

FIG. 10 is a schematic view illustrating a variation of the flow path structure 100 in the present modification. In addition, the main flow direction of the flow flowing in from the second flow path 2 is indicated by an arrow. In the example illustrated in FIG. 10(a), a location (indicated by a broken line in the drawing) at which the distances from the two flow path side surfaces of the second flow path 2 matches the intersection of the extended lines of the wall surfaces of the first flow path 1 and the third flow path 3 intersect with each other. With this structure as a reference, a case where the position of the second flow path 2 is shifted toward the first flow path 1 or the third flow path 3 will be considered. FIG. 10(b) illustrates a state where the second flow path 2 is shifted to the first flow path 1 side. In FIG. 10(b), the end portion of the side surface of the second flow path 2 farther from the first flow path 1 coincides with a point (corner) where the extended lines of the wall surfaces of the first flow path 1 and the third flow path 3 intersect with each other. At this time, most of the flow from the second flow path 2 does not impinge on the side surface of the first flow path 1 as in the case of FIG. 10(a), and a separation vortex is formed in the third flow path 3. FIG. 10(c) illustrates a state where the second flow path 2 is further shifted to the first flow path 1 side. When the second flow path 2 is separated from the mixing region 13, the flow from the second flow path 2 impinges on the wall surface of the first flow path 1, and the separation vortex is disturbed. Therefore, in the case of a structure in which the second flow path 2 is shifted toward the first flow path 1 side, the shift amount is preferably limited to approximately half the flow path width of the second flow path 2 such that the distance L from the second opening 12 to the corner does not exceed this extent (as shown in FIG. 10(c)). FIG. 10(d) illustrates a state where the second flow path 2 is shifted to the third flow path 3 side. In FIG. 10(d), the end portion of the side surface of the second flow path 2 closer to the first flow path 1 coincides with a point (corner) where the extended lines of the wall surfaces of the first flow path 1 and the third flow path 3 intersect with each other. When the second flow path 2 is largely shifted toward the third flow path 3 side and separated from the mixing region 13, the flow from the second flow path 2 flows closely along the side surface of the third flow path 3, and a separation vortex is hardly formed. Therefore, in the case of a structure in which the second flow path 2 is shifted toward the third flow path 3 side, the shift amount is preferably limited to the extent that the second opening 12 includes a corner (the extent illustrated in FIG. 10(d)).

### Second Embodiment

Components common to those of the first embodiment will be given the same reference numerals, and a second embodiment will be described below while omitting description of components having the same configuration and function.

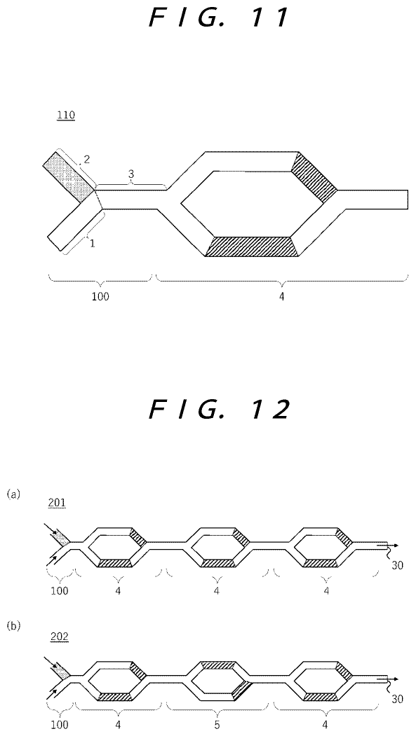

FIG. 11 is an example of a plan view of a flow path structure 110. The flow path structure 110 is a larger flow path structure partially including the flow path structure 100. The flow path structure 110 further includes a flow path having a branch structure and a merging structure on the opposite side of the first flow path 1 and the second flow path 2 with respect to the third flow path 3. The flow path structure 110 includes a mixing flow path 4 in addition to the flow path structure 100. The mixing flow path 4 includes a branch portion where one flow path branches into two or more flow paths, and a merging portion where the branched flow paths merge again. In FIG. 11, a section of the mixing flow path 4 where the flow path depth is shallower than other sections and the top surface and the bottom surface are parallel is indicated by hatching. Hereinafter, in the present specification, a region having a shallower flow path depth than other sections will be described as a “shallow portion”. By using a flow path including a shallow portion similar to the mixing flow path 4 in the drawing, a transverse vortex is generated in the flow path, and the fluid can be further mixed and stirred. The bottom surface or the top surface of the second flow path 2 is inclined in the same manner as that described in the first example, and the liquid injected into each of the first flow path 1 and the second flow path 2 can be quickly mixed in the third flow path 3.

Although the mixing flow path 4 in which the predetermined section is a shallow portion is illustrated in the drawing, the shape of the mixing flow path 4 is not limited thereto, and for example, the section between the branch portion and the merging portion may have a slope shape along the tube axis of the flow path, or the section between the branch portion and the merging portion may have a curved structure.

### Third Embodiment

Components common to those of the first and second embodiments will be given the same reference numerals, and a third embodiment will be described below while omitting description of components having the same configuration and function.

FIG. 12 is an example of a schematic view when the flow path structure 100, the plurality of mixing flow paths 4, and the like are used in combination. Hereinafter, a unit formed by combining a plurality of mixing flow paths 4 and the flow path structures corresponding to the mixing flow paths 4 will be described as a “flow path structure unit”. In FIG. 12(a), an example in which three mixing flow paths 4 are connected in series will be described. As illustrated in the drawing, a flow path structure unit 201 may include, on the downstream side, an outlet 30 through which the liquid can flow out to the outside of the flow path structure unit 201. By connecting the plurality of mixing flow paths 4 in series in this manner, it is possible to repeatedly generate vortices and to achieve more rapid mixing of different types of liquids.

A flow path structure unit 202 illustrated in FIG. 10(b) includes a mixing flow path 5, unlike the flow path structure unit 201 illustrated in FIG. 12(a). The position of the shallow portion of the mixing flow path 5 is different from that of the mixing flow path 4, but other configurations are the same as those of the mixing flow path 4. It can also be said that the mixing flow path 5 has a line symmetrical structure with respect to the mixing flow path 4 with the third flow path 3 as an axis. The flow path structure unit 202 having a structure in which the mixing flow path 4 and the mixing flow path 5 which are line-symmetrical with the third flow path 3 as an axis are alternately arranged can more uniformly mix the two types of liquids flowing in from the flow path structure 100.

Although the number of mixing flow paths included in the flow path structure units 201 and 202 illustrated in the drawing is three, the number of mixing flow paths may be one or two, and may be four or more.

### First Modification

Components common to those of the second embodiment will be given the same reference numerals, and modifications will be described below while omitting description of components having the same configuration and function.

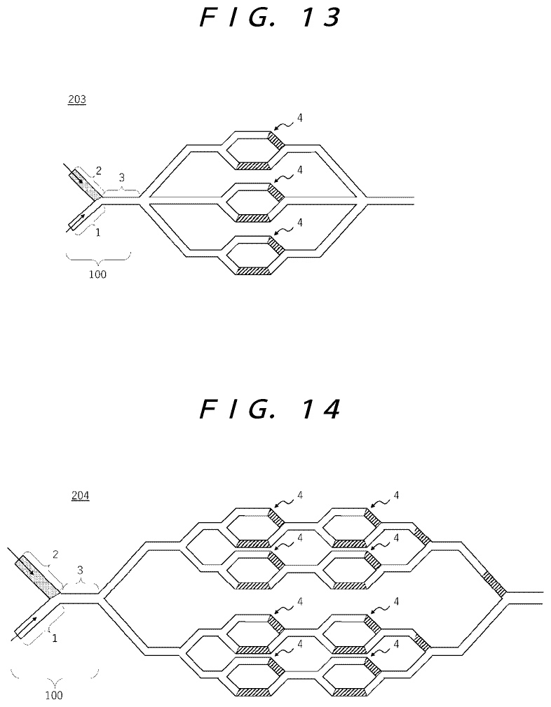

FIG. 13 is an example of a schematic view when the flow path structure 100 and the plurality of mixing flow paths 4 are used in combination. In FIG. 13, an example in which three mixing flow paths 4 are connected in parallel will be described. A flow path structure unit 203 has a branch portion on the downstream side of the third flow path 3, and each of the branch portions includes the mixing flow path 4 at the branched end thereof. The liquid is branched on the upstream side, passed through the plurality of mixing flow paths 4, and then merged into one flow path again on the downstream side. According to this arrangement, it is possible to reduce the resistance of liquid feeding even in a case where the flow rate is large as compared with the case of being arranged in series. In a case where a liquid feeding pump is used to feed liquid toward the flow path structure unit 203, the load on the pump can be reduced.

### Second Modification

Components common to those of the second embodiment will be given the same reference numerals, and modifications will be described below while omitting description of components having the same configuration and function.

FIG. 14 is an example of a schematic view when the flow path structure 100 and the plurality of mixing flow paths 4 are used in combination. FIG. 14 illustrates a structure in which the series arrangement and the parallel arrangement are used in combination. In that case, it is possible to adjust the resistance of liquid feeding and to enhance the effect of stirring and mixing. For example, a flow path structure unit 204 illustrated in FIG. 14 includes four sets of flow path structure units each including two mixing flow paths 4 arranged in series, and these four sets of flow path structure units are arranged in parallel. In addition, in the part where the fluids merge downstream of the flow path structure units arranged in parallel, it is preferable that at least one flow path has a structure in which the flow path depth changes in the flow path width direction similar to the second flow path 2 in order to promote mixing and stirring. The flow path structure using both the series arrangement and the parallel arrangement is not limited to the example illustrated in FIG. 14, and can be modified according to the type or application of the fluid.

### Fourth Embodiment

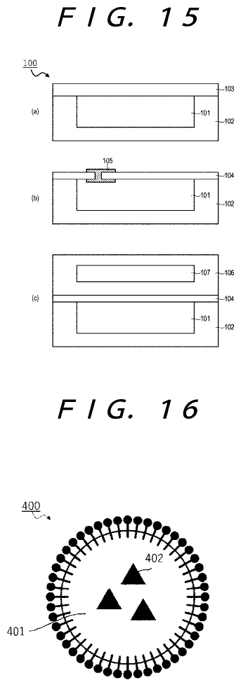

In the present embodiment, a method for producing the flow path structure 100 and the flow path structure units 201 to 204 (hereinafter representatively referred to as “flow path structure 100”) described in the first to third embodiments will be described below with reference to FIG. 15. As illustrated in FIG. 15(a), the flow path structure 100 includes, for example, a substrate 102 in which a groove 101 functioning as a flow path is formed, and a plate-like lid portion 103 joined to the substrate 102 to close a top surface of the groove 101.

The material of the substrate 102 may be appropriately selected from resins such as acrylic, polyethylene, polypropylene, and polycarbonate, glass, ceramics, metal, and the like according to the application. For example, when the flow path structure 100 is for medical use, a cycloolefin polymer (COP) or the like is also a preferred example. Ceramics such as glass and quartz are preferable from the viewpoint of stability when reused many times, and a metal having a surface subjected to a treatment for corrosion resistance may be used when the temperature and the like are adjusted. The groove 101 corresponds to the first flow path 1, the second flow path 2, and the third flow path 3, and for example, a bulk solid can be formed by press working or cutting using a mold. In the location corresponding to the shallow portion, the groove 101 may be formed or cut shallower than other parts.

As the material of the lid portion 103, for example, the same material as that described for the substrate 102 can be used. The lid portion 103 may have, for example, a plate shape. Alternatively, as illustrated in FIG. 15(b), a thin film-like lid portion 104 may be used.

It is also possible to attach a sensor terminal 105 for monitoring the state of the fluid to the film-like lid portion 104. Alternatively, it is also possible to impart various functions or properties such as high thermal conductivity or a function (not illustrated) of performing a specific treatment on a specific substance with respect to the film-like lid portion 104.

In a case where there is a concern that the film-like lid portion 104 may swell due to the internal pressure, as illustrated in FIG. 22(c), the swell may be suppressed by pressing a pressing plate 106 from above the film-like lid portion 104. The pressing plate 106 may include a heat medium flow path 107 for heat exchange disposed therein, an electric terminal (not illustrated) having a sensor function, or the like.

Thus, the flow path structure 100 can be produced by a simple procedure in which the groove 101 is formed in the substrate 102 and the lid portion 103 or the film-like lid portion 104 is joined. Therefore, for example, it is unnecessary to form grooves in both the substrate 102 and the lid portion 103 and to precisely align the substrate 102 and the lid portion 103, and thus mass productivity is extremely high.

Further, the depth of the groove 101 at the location where the flow path depth becomes smaller may be set to the same depth as the other parts, and the film-like lid portion 104 of which the thickness changes depending on the position may be attached to the corresponding location to form a flow path of which the flow path depth changes. That is, in the flow path formed in this manner, the thickness of the top surface changes depending on the position, accordingly, the flow path depth changes. Even in such a structure, highly uniform mixing can be realized as in a structure in which the shape of the bottom surface changes depending on the position.

### Fifth Embodiment

In the present embodiment, a fluid stirring method is provided. The fluid stirring method includes flowing a fluid to be stirred into the flow path structure 100 of the embodiment. According to the fluid stirring method, the fluid can be further mixed and stirred by using the flow path structure 100 of the embodiment.

In a case where the flow path structure 100 of the first to fourth embodiments is used, the present method includes causing a liquid to flow to pass through the first flow path 1 or the second flow path 2 and flow through the third flow path 3. Furthermore, in the present method, the liquid flowing in the flow path may be two or more different types of fluids, and according to the flow path structure 100 of the first to fourth embodiments, these fluids can be quickly mixed and stirred.

### Sixth Embodiment

In the present embodiment, a method for producing a lipid particle 400 encapsulating a drug 402 using the flow path structure 100 of the embodiment will be described.

First, the lipid particle 400 produced by the present method will be described. FIG. 16 is a diagram illustrating an example of the lipid particle of the present embodiment. As illustrated in FIG. 16, the lipid particle 400 includes a lipid membrane formed by arranging lipid molecules, and has a substantially hollow spherical shape. The drug 402 is encapsulated in a lumen 401 of the lipid particle 400. The lipid particle 400 may be used, for example, to deliver the drug 402 into cells.

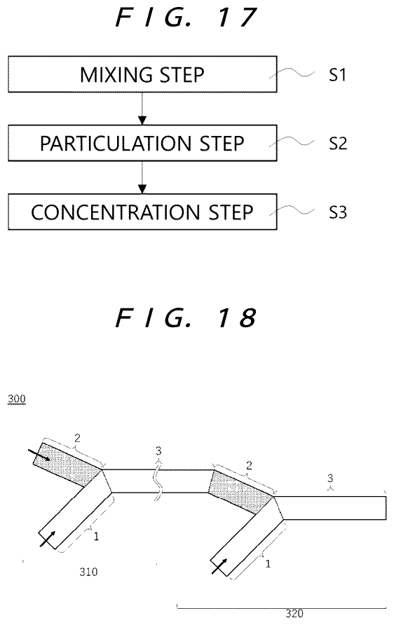

FIG. 17 is a flowchart illustrating an example of a method for producing the lipid particles 400. A method for producing the lipid particles 400 by using the flow path structure 100 of the embodiment includes: a step (mixing step S1) of flowing a first solution containing a lipid which is a material of the lipid particles 400 in an organic solvent from one inflow port (first inflow port) positioned on an upstream side of the first flow path 1 and flowing a second solution containing the drug 402 in an aqueous solvent from another inflow port (second inflow port) positioned on an upstream side of the first flow path 1 to mix the first solution and the second solution to obtain a mixed solution; a step (particle formation step S2) of reducing the concentration of the organic solvent in the mixed solution to form the lipid into particles and generate the lipid particles 400 encapsulating the drug 402; and a step (concentration step S3) of concentrating the solution of the lipid particles 400. FIG. 18 is a view illustrating an example of a flow path structure 300 used in the method for producing the lipid particles of the present embodiment. The present production method can be performed using, for example, the flow path structure 300. The mixing step S1 is performed in a region 310 in the drawing, and the particle formation step S2 is performed in a region 320.

Hereinafter, an example of a procedure of the present production method will be described.

First, a first solution and a second solution are prepared. The first solution contains a lipid in an organic solvent. The lipid is a material constituting the lipid particle 400. The second solution contains the drug 402 in an aqueous solvent.

### Mixing Step S1

The first solution and the second solution are mixed. As the second solution, the second solution can be prepared by mixing the drug 402 with any of the aqueous solvents selected according to the type thereof. The drug 402 is a nucleic acid or the like, and the drug 402 that is not a nucleic acid includes, for example, a protein, a peptide, an amino acid, another organic compound, an inorganic compound, or the like as an active component. The drug 402 may be, for example, a therapeutic agent or diagnostic agent for a disease. However, the drug 402 is not limited thereto, and may be any substance as long as the substance can be encapsulated in the lipid particle 400.

If necessary, the drug 402 may further contain a reagent such as, for example, a pH adjusting agent, an osmotic pressure adjusting agent, and/or a drug activating agent. The pH adjusting agent is, for example, an organic acid such as citric acid and a salt thereof. The osmotic pressure adjusting agent is a sugar, an amino acid, or the like.

The first solution can be produced by mixing a lipid and an organic solvent. The lipid may be, for example, a lipid of a main component of a biological membrane. The lipid may be artificially synthesized. The lipid can include, for example, a base lipid such as a phospholipid or a sphingolipid, for example, diacylphosphatidylcholine, diacylphosphatidylethanolamine, ceramide, sphingomyelin, dihydrosphingomyelin, kephalin, or cerebroside, or a combination thereof.

The organic solvent of the first solution is, for example, ethanol, methanol, isopropyl alcohol, ether, chloroform, benzene, acetone, or the like.

Mixing of the first solution and the second solution is performed in the region 310 illustrated in FIG. 18. In FIG. 18, the structure between the region 310 and the region 320 is omitted, but any flow path structure unit may be included between the actual region 310 and the region 320.

### Particle Formation Step S2

Next, in the particle formation step S2, the concentration of the organic solvent in the mixed solution is reduced. For example, it is preferable to relatively lower the organic solvent concentration by adding a large amount of the aqueous solution to the mixed solution. For example, three times the amount of the aqueous solution of the mixed solution is added to the mixed solution. As the aqueous solution, the same aqueous solvent as that used for the first solution can be used. By decreasing the concentration of the organic solvent, the lipid can be formed into particles, and the lipid particle 400 encapsulating the drug 402 can be generated. As a result, a lipid particle solution containing the lipid particles 400 is obtained.

The region 320 is, for example, a Y-shaped flow path. The upstream end of one of the branched Y-shaped flow paths is connected to the most downstream end of the region 310, from which the mixed solution is supplied. The upstream end of the other flow path includes, for example, an aqueous solution inflow port, and the aqueous solution flows from the aqueous solution inflow port. As a result, the aqueous solution is mixed with the mixed solution in the region 320 where the mixed solution having passed through the region 310 and the aqueous solution injected from the aqueous solution inflow port merge with each other. As a result, the lipid is formed into particles, the lipid particle 400 encapsulating the drug 402 is generated, and a lipid particle solution containing the lipid particle 400 is obtained.

The particle formation step S2 is not necessarily performed using a flow path, and for example, an aqueous solution may be added to the mixed solution collected in the container.

In this manner, the lipid particles 400 can be produced.

### Concentration Step S3

The method for producing lipid particles of the embodiment may further include concentrating the lipid particle solution as necessary (concentration step S3). The concentration is performed, for example, by removing a part of the solvent and/or excess lipid and the drug 402 from the lipid particle solution. The concentration can be performed, for example, by ultrafiltration. For ultrafiltration, for example, an ultrafiltration filter having a pore diameter of 2 nm to 100 nm is preferably used. For example, Amicon (registered trademark) Ultra-15 (Merck) or the like can be used as the filter.

The flow of the fluid in the flow path, the injection of the fluid into the flow path, the extraction of the fluid from the tank, and/or the accommodation of the lipid particle solution into the container can be performed by, for example, a pump or an extrusion mechanism configured and controlled to automatically perform these operations.

When the drug 402 is a nucleic acid, a condensation step of condensing the nucleic acid (drug 402) may be performed before the mixing step S1. The nucleic acid drug 402 is, for example, a nucleic acid containing DNA, RNA, and/or other nucleotides, and may be, for example, mRNA of a specific gene, DNA encoding a gene, DNA containing a gene expression cassette containing a gene and other sequences for expressing a gene such as a promoter, a vector, or the like.

Although the embodiments of the present invention have been described above, these embodiments are presented as examples, and are not intended to limit the scope of the invention. These novel embodiments can be implemented in various other forms, and various omissions, substitutions, and changes can be made without departing from the gist of the invention. These embodiments are included in the scope and gist of the invention and are included in the invention described in the claims and the equivalent scope thereof.

In addition, in the present specification, it has been described that the tube axis direction and the direction of the main flow of the flow path structure are as indicated by arrows in FIG. 1, but the tube axis direction and the direction of the main flow of the flow path structure are not limited thereto.

In addition, the “tube axis” described in the present specification may be, for example, a central axis of a flow path. However, this definition does not limit the interpretation of “tube axis” in a narrow sense, and should be interpreted as appropriate without impairing the gist of the invention described in the embodiment.

In addition, the embodiments and modifications described in the present specification can be arbitrarily combined.

Furthermore, the present disclosure includes examples according to the following supplementary notes.

### Supplementary Note 1

A flow path structure including:
a first flow path;
a second flow path connected to the first flow path; and
a third flow path connected to the first flow path and the second flow path, in which
when a distance between a top surface and a bottom surface of a flow path is a flow path depth, a flow path depth of the second flow path at an opening where the first flow path is connected to the second flow path is not constant, and a maximum value of the flow path depth of the second flow path at the opening is smaller than a flow path depth of the first flow path.

### Supplementary Note 2

The flow path structure according to Supplementary Note 1, in which
the flow path depth of the second flow path changes at a constant inclination.

### Supplementary Note 3

The flow path structure according to any one of Supplementary Note 1 and 2, in which
the maximum value of the flow path depth of the second flow path is ½ or less of the flow path depth of the first flow path.

### Supplementary Note 4

The flow path structure according to any one of Supplementary Notes 1 to 3, in which top surfaces of the first flow path, the second flow path, and the third flow path are included in a single plane.

### Supplementary Note 5

The flow path structure according to any one of Supplementary Notes 1 to 4, in which the first flow path, the second flow path, and the third flow path are formed by cutting or pressing a bulk solid.

### Supplementary Note 6

The flow path structure according to any one of Supplementary Notes 1 to 5, in which a flow path depth of the second flow path at a location where a flow path side surface of the second flow path is in contact with a flow path side surface of the first flow path is larger than a flow path depth of the second flow path at a location where a flow path side surface of the second flow path is in contact with a flow path side surface of the third flow path.

### Supplementary Note 7

The flow path structure according to any one of Supplementary Notes 1 to 6, in which
the first flow path and the second flow path are symmetrical with respect to the third flow path.

### Supplementary Note 8

The flow path structure according to any one of claims 1 to 7, in which
each of an angle formed by a side surface of the first flow path and a side surface of the third flow path and an angle formed by a side surface of the second flow path and a side surface of the third flow path is 30° or more and 60° or less.

### Supplementary Note 9

The flow path structure according to any one of Supplementary Notes 1 to 8, in which
the flow path depth is equal to or less than flow path widths of the first flow path, the second flow path, and the third flow path.

### Supplementary Note 10

The flow path structure according to any one of Supplementary Notes 1 to 9, in which
a cross-sectional area of the flow path, included in the flow path structure, perpendicular to a tube axis direction of the flow path is 100 mm² or less.

### Supplementary Note 11

The flow path structure according to any one of Supplementary Notes 1 to 10, further including:
a flow path in which the first flow path and the second flow path are provided at one end and at least one of a branch structure and a merging structure is provided at multiple ends.

### Supplementary Note 12

A fluid structure unit including:
the flow path structure according to Supplementary Note 1; and
a flow path having a plurality of branch structures and a merging structure.

### Supplementary Note 13

A method for producing lipid particles encapsulating a drug using the flow path structure according to any one of Supplementary Notes 1 to 10, the method including:
a step of flowing a first solution containing a lipid to be a material of the lipid particles in an organic solvent from a first inflow port positioned on an upstream side of the first flow path and flowing a second solution containing the drug in an aqueous solvent from a second inflow port positioned on an upstream side of the first flow path to mix the first solution and the second solution to obtain a mixed solution; and
a step of reducing the concentration of the organic solvent in the mixed solution to form the lipid into particles and generate the lipid particles encapsulating the drug.

What is claimed is:

1. A flow path structure comprising:
a first flow path;
a second flow path connected to the first flow path; and
a third flow path connected to the first flow path and the second flow path, wherein
when a distance between a top surface and a bottom surface of a flow path is a flow path depth, a flow path depth of the second flow path at an opening where the first flow path is connected to the second flow path is not constant, and a maximum value of the flow path depth of the second flow path at the opening is smaller than a flow path depth of the first flow path.

2. The flow path structure according to claim 1, wherein
the flow path depth of the second flow path changes at a constant inclination.

3. The flow path structure according to claim 1, wherein
the maximum value of the flow path depth of the second flow path is ½ or less of the flow path depth of the first flow path.

4. The flow path structure according to claim 1, wherein
top surfaces of the first flow path, the second flow path, and the third flow path are included in a single plane.

5. The flow path structure according to claim 1, wherein
the first flow path, the second flow path, and the third flow path are formed by cutting or pressing a bulk solid.

6. The flow path structure according to claim 1, wherein
a flow path depth of the second flow path at a location where a flow path side surface of the second flow path is in contact with a flow path side surface of the first flow path is larger than a flow path depth of the second flow path at a location where a flow path side surface of the second flow path is in contact with a flow path side surface of the third flow path.

7. The flow path structure according to claim 1, wherein
the first flow path and the second flow path are symmetrical with respect to the third flow path.

8. The flow path structure according to claim 1, wherein
each of an angle formed by a side surface of the first flow path and a side surface of the third flow path and an angle formed by a side surface of the second flow path and a side surface of the third flow path is 30° or more and 60° or less.

9. The flow path structure according to claim 1, wherein
the flow path depth is equal to or less than flow path widths of the first flow path, the second flow path, and the third flow path.

10. The flow path structure according to claim 1, wherein
a cross-sectional area of the flow path, included in the flow path structure, perpendicular to a tube axis direction of the flow path is 100 mm² or less.

11. The flow path structure according to claim 1, further comprising:
a flow path in which the first flow path and the second flow path are provided at one end and at least one of a branch structure and a merging structure is provided at the other end.

12. A fluid structure unit comprising:
the flow path structure according to claim 1; and
a flow path having a plurality of branch structures and a merging structure.

13. A method for producing lipid particles encapsulating a drug using the flow path structure according to claim 1, the method comprising:
a step of flowing a first solution containing a lipid to be a material of the lipid particles in an organic solvent from a first inflow port positioned on an upstream side of the first flow path and flowing a second solution containing the drug in an aqueous solvent from a second inflow port positioned on an upstream side of the first flow path to mix the first solution and the second solution to obtain a mixed solution; and
a step of reducing the concentration of the organic solvent in the mixed solution to form the lipid into particles and generate the lipid particles encapsulating the drug.
